(19) United States
(12) Patent Application Publication
(10) Pub. No.: US 2021/0172161 A1
(43) Pub. Date: Jun. 10, 2021

(54) DEVICE FOR THE ON-SITE ANALYSIS OF EXCREMENTS, METHOD FOR OPERATING SUCH A DEVICE, ARRANGEMENT CONSISTING OF A TOILET AND SUCH A DEVICE, AS WELL AS SAMPLE CARRIER

(71) Applicant: Medipee GmbH, Moers (DE)

(72) Inventors: Thomas Prokopp, Bonn (DE); Frank Willems, Moers (DE); Paul Bandi, Kamp-Lintfort (DE); Eric Kruissekbrink, Winterswijk (NL)

(21) Appl. No.: 16/763,780

(22) PCT Filed: Nov. 12, 2018
(86) PCT No.: PCT/DE2018/000328

§ 371 Date: May 13, 2020

(30) Foreign Application Priority Data
Nov. 14, 2017 (DE) 10 2017 010 519.7

(51) Int. Cl. E03D 9/00; E03D 9/10; G01N 33/483

(52) U.S. Cl. CPC E03D 9/002; G01N 33/483; E03D 9/10

(57) ABSTRACT

The invention relates to a device for the on-site analysis of excrements, comprising a housing, a removal device, by means of which a sample of excrement can be removed, in which the removal device comprises an arm element for a sample carrier, which is movable relative to the housing in the axial movement direction, said arm element being guided at least partially in a guide part in such a way that it can be retracted and extended, a feeding device which supports a new sample carrier for the removal device, and an analysis device which at least partially analyzes the sample, wherein the device is characterized in that the guide part comprises a contamination region with a guide path for guiding the moveable arm element, and also a clean region, in which a new sample carrier can be arranged at least partially next to the guide path.

The invention relates on the one hand to a device for the on-site analysis of excrements, comprising a housing, a removal device, by means of which a sample of excrement can be removed, in which the removal device comprises an arm element for a sample carrier, which is movable relative to the housing and which is guided at least partially in a guide part in such a way that it can be retracted and extended, a feeding device, by means of which a new sample carrier can be provided for the removal device, and an analysis device, by means of which the removed sample can be at least partially analyzed.

The invention on the other hand relates to a device for the on-site analysis of excrements, comprising a housing, a removal device, by means of which a sample of excrement can be removed, in which the removal device comprises an arm element for a sample carrier, which is movable relative to the housing and which is guided at least partially in a guide part in such a way that it can be retracted and extended, and an analysis device, by means of which the removed sample can be at least partially analyzed.

The invention also relates to a method for operating a device for the on-site analysis of excrements, in which a sample carrier is moved for sampling linearly relative to a sensor unit by means of an axially movable arm element, in which the sample carrier is held on a holder of the axially movable arm element, and in which a sample of excrement carried by the sample carrier is at least partially analyzed on the device.

The invention further relates to an arrangement consisting of a toilet, a urinal or the like, and a device for the on-site analysis of excrements.

The invention also relates to a sample carrier for an on-site analysis of excrements.

It is known that, by means of analyses of urine and/or stool samples, at least first insights into the health status of an organism, particularly a human being, can be provided. However, in order to obtain sufficiently meaningful analysis information, it is usually necessary to safely place urine and/or stool samples removed, e.g., from a toilet, in a transport container in order to send said container containing the sample to an analysis laboratory. This is not only inconvenient, but can also take longer before corresponding analysis results are available. However, it is particularly disadvantageous that a close monitoring in the sense of continuous preventive examinations does usually not take place due to the considerable effort involved. In this respect, it is desirable to create a method that facilitates the analysis of excrements.

From WO 2017/021452 A1, particularly a device for the mobile analysis of excrements in a toilet is known, so that a daily analysis of urine and/or stool samples can be carried out on site, i.e., immediately at the location of the sampling.

The problem addressed by the invention is that of developing particularly a device of the type in question for analyzing excrements, especially human excrements, so that the device works more reliably.

According to a first aspect of the invention, the problem addressed by the invention is solved by a device for the on-site analysis of excrements, comprising a housing, a removal device, by means of which a sample of excrement can be removed, in which the removal device comprises an arm element for a sample carrier, which is movable relative to the housing and which is guided at least partially in a guide part in such a way that it can be retracted and extended, a feeding device, by means of which a new sample carrier can be provided for the removal device, and an analysis device, by means of which the removed sample can be at least partially analyzed, wherein the device is characterized in that the guide part comprises a contamination region with a guide path for guiding the movable arm element, and also a clean region, in which a new sample carrier can be arranged at least partially next to the guide path.

Due to the fact that the guide part has a contamination region and a clean region, the device can be operated more reliably.

In particular, the risk that excrements, particularly excrements from another user, inadvertently contaminate a new sample carrier while it is moved along the guide part for sampling, namely when the movable arm element with the new sample carrier for a new sampling is moved axially along the guide path, can be completely eliminated or at least significantly reduced. This, in turn, reduces the risk of falsified analysis results.

In this respect, a constant mobile monitoring using urine and/or stool samples is even easier and more reliable.

In the sense of the invention, the movable arm element is movably mounted relative to the guide part in an axial movement direction, wherein the axial movement direction is oriented along the longitudinal extension of the guide part.

More specifically, the movable arm element for removing a new sample of excrements together with a new, i.e., unused, sample carrier can be at least partially extended axially in the axial extension direction from the guide part.

Furthermore, the movable arm element, together with the sample carrier moistened with urine or stool, i.e., the used sample carrier, can be axially retracted again in the axial retraction direction.

In this respect, the term “axial” refers to the actual movement direction of the movable arm element or the sample carrier.

The term “removal device” in the sense of the invention refers to any device, by means of which a sample of excrement can be removed and fed to the device for analysis.

In the present case, the term “feeding device” describes a device, by means of which a new sample carrier can be transferred to the movable arm element.

In the present case, the term “analysis device” refers to all devices that are suitable for sensorially detecting and analyzing a sample of excrement.

For this purpose, the analysis device also comprises at least one sensor, preferably an entire sensor unit with a multiplicity of identical or, preferably, different sensors.

In the sense of the invention, the term “contamination region” describes the region of the guide part, in which the movable arm element is guided. In this respect, the guide path of the guide part is also located in said contamination region. In this case, it cannot be completely ruled out that the movable arm element comes into contact with excrement.

Said contamination region essentially extends from a guide path opening arranged at the free end of the guide part and a transfer region, in which a new sample carrier is transferred from the feeding device to a holder located on the arm element.

In the present case, the term “clean region” describes a region of the guide part, which neither the movable arm element nor a used sample carrier can reach.

Said clean region describes particularly a sector on the guide part between a sensor unit arranged at the free end of the guide part and the transfer region, in which a new sample carrier is transferred from the feeding device to a holder located on the arm element.

The free end of the guide part denotes the end of the guide part, from which the movable arm element is extended from the guide part for sampling. The sensor unit is also located at said free end.

In the sense of the invention, the term “sensor unit” describes a device which at least essentially comprises at least one sensor, preferably a plurality of sensors, which sensorially detect a sample adhering to the sample carrier for analysis.

In the present case, such a sensor unit can be constructed in a wide variety of ways in order to be able to at least temporarily place a sample carrier opposite the sensor unit and/or to be arranged or formed, for example, on the guide part in accordance with the imposed requirements.

It goes without saying that different sensors can be provided for this purpose.

For example, the sensor unit comprises a color sensor for detecting a color change with respect to the sample carrier, whereby a first analysis in the sense of the invention can already be performed.

Further data of the sensor unit can be transmitted cumulatively or alternatively to an analysis unit, which can be located, for example, in the housing of the device and/or also on the guide part of the device.

Furthermore, it is possible for data to be transmitted cumulatively or alternatively from the sensor unit and/or from the analysis unit to an external analysis unit. For this purpose, for example, a data processing and/or analysis application running on a smartphone or the like can be taken into consideration.

The transmission can be wireless or wired, wherein the transmission between the device and an external data processing and/or analysis application or the like takes place preferably in a wireless manner.

For the analysis, the analysis device, particularly the sensor unit and/or an additional analysis unit, advantageously comprises a suitable microcontroller or the like, by means of which, in the simplest embodiment, e.g., a color change on the sample carrier can be analyzed.

In this case, it is conceivable that, depending on the excrement to be analyzed, at least some of the sensors can be switched off or on, resulting in an energetically more efficient operation of the device.

The “movable arm element” can be designed in a variety of ways, for example, as a telescopic arm element, with arm segments that can be retracted into one another, or the like.

However, the present arm element preferably comprises a material strip that can be rolled up or unrolled, and which can be unrolled from a coil for extending the arm element and rolled up again onto the coil for retracting the arm element.

The term “sample carrier” describes a piece of material that is suitable for receiving excrement, for example, an indicator strip or lamella, or the like.

At this point, it must already be noted that such a sample carrier can be designed in a variety of ways in order to be used advantageously in the sense of the present invention. For example, the sample carrier is also at least partially transparent to visible light.

According to a second aspect of the invention, the present problem is insofar also solved by a sample carrier for an on-site analysis of excrements, comprising a main part, wherein the sample body has individual sample analysis fields with different geometrical shapes.

As a result, it is possible to particularly design different indicator surfaces on the sample carrier, so that several analyses can be performed independently of one another.

By means of a sample carrier thus designed, the present device can operate even more efficiently.

In this context, the problem addressed by the invention is also solved by a sample carrier for an on-site analysis of excrements, comprising a main part, wherein sample analysis fields are arranged both on the front side and on the rear side of the main part. If sample analysis fields are arranged on both sides of the main part, the number of analyses that can be carried out simultaneously can be significantly increased.

Corresponding devices for the on-site analysis of excrements and related methods for operating such devices can also be significantly developed by the sample carriers claimed above. For example, by using corresponding sample carriers, the number of sensor units on devices can be increased in a meaningful manner, so that related processes can be carried out even faster.

In the case of conventional urine test strips, the test materials (reagent or absorbent paper) are always applied to the carrier materials (herein: main part) from one side only. In the present invention, sample analysis fields are applied to the carrier material from both sides. This saves space and reduces the demand for carrier material and also sample carriers.

In addition, with regard to a single sample carrier, more analyses can be performed simultaneously without hesitation if individual sample analysis fields have different indicators.

Furthermore, a risk of a mutual influencing of different indicators can be reduced if individual sample analysis fields are arranged on the main part at a distance from one another.

For the purpose of further optimization, the shape of the sample analysis fields, which up to now has mainly been a square shape of 5 mm×5 mm, can also be changed.

In this respect, a further embodiment provides that individual sample analysis fields are designed to be circular.

However, other different geometric shapes are also conceivable, such as smaller square areas, circular areas, oval areas, or the like.

If the main part and individual sample analysis fields are at least partially translucent, the variety of analysis methods can be significantly increased with the use of a suitably equipped device. For example, a transmitted light measurement or the like could be taken into consideration.

If the sample carrier is provided by the feeding device and has not yet been contaminated with excrement, it is referred to as a new sample carrier in the sense of the invention.

If, however, a sample carrier is contaminated with excrement, it is in the present case referred to as a used sample carrier.

The term “excrement” in the sense of the invention describes body excretions, such as urine and stool.

In the sense of the invention, the term “on-site analysis” describes that an analysis of excrements can be performed at least partially or preferably entirely at the place of excretion, for example, at a toilet, a urinal, or the like.

In other words, this means that any type of excrement to be examined does not have to be sent elaborately to a remote laboratory for analysis.

It goes without saying that data or information obtained on-site for further evaluation or storage can also be transmitted to external devices, such as a data carrier, etc.

For removing a desired sample, the movable arm element of the removal device is in any case extended from the guide part to such an extent that a new, fresh sample carrier can come into contact with the respective excrement.

The sample carrier contaminated with the excrement is then retracted into the guide part for analysis in order to be able to be examined therein by means of sensors of a sensor unit of the device.

A preferred embodiment provides that the clean region of the guide part is arranged on the side next to the contamination region of the guide part, wherein the clean region and the contamination region are spatially separated from one another by a separation, preferably by a rigid partition. The clean region and the contamination region, particularly the guide path of the movable arm element, are thus very clearly separated from one another, so that the risk can be reduced that excrement adhering to the arm element can enter the clean room and in there contaminate a new sample carrier with old excrement, possibly from another person.

In order to make it possible that the sample carrier can still be held across the partition with its first half on the holder of the movable arm element (contamination region) and that the other half can be fed contamination-free through the guide part (clean region) to the new sample, it is expedient if a slit-like opening is arranged in the region of the partition, through which a new sample carrier can be arranged both in the contamination region and in the clean region.

Furthermore, it is advantageous if the clean region of the guide part, as seen looking in the retraction direction of the movable arm element, is arranged axially behind a release unit for releasing a used sample carrier from the movable arm element. This can ensure that the used sample carrier is reliably released from the retracting arm element before reaching the clean room, so that a contamination of the clean room can be ruled out if the device is operated properly.

It goes without saying that said slit-like opening can be designed in different ways. If necessary, bristle strips are exclusively or additionally provided on the partition in order to realize a partition which is essentially only permeable to the sample carrier.

It is possible to design the slit-like opening in such a way that the sample carrier can be guided above and below in the region of the partition, resulting in a certain height guidance for the sample carrier within the guide part. As a result, the sample carriers can always be held in front of the sensors at possibly the same distance.

According to a third aspect of the invention, the problem addressed by the invention is solved cumulatively or alternatively by a device for the on-site analysis of excrements, comprising a housing, a removal device, by means of which a sample of excrement can be removed, in which the removal device comprises an arm element for a sample carrier, which is movable relative to the housing in the axial movement direction, said arm element being guided at least partially in a guide part in such a way that it can be retracted and extended, and an analysis device, by means of which the removed sample can be at least partially analyzed, wherein the device is characterized by a sensor unit having a transmitted light measuring apparatus for transilluminating the sample carrier.

With such a transmitted light measuring apparatus, particularly precise examination results can be achieved, particularly a qualified examination and determination of or on excrements.

In particular, a measurement and evaluation of an indicator material can advantageously be performed by means of a transmitted light measurement.

In particular, optical path lengths for a light emitted from a light source through a sample are significantly increased by means of a correspondingly designed sensor unit or the transmitted light measuring apparatus and a transmitted light measurement associated therewith.

In this case, a higher information density is advantageously obtained because the sample carrier or a corresponding test material is transilluminated, and the sample carrier is not only irradiated on the surface, as has been customary until now, for example, in reflection photometry, in which properties of a radiation, which is reflected by a surface illuminated by a light source, are measured by means of a reflection photometer.

In particular, the already described indicator strips or lamellas, etc., can be used as test material, but materials especially provided for this purpose can also be used.

A very simple form of transmitted light measurement can be performed, for example, by saturating a simple receiving material, for example, a cellulose-based material (filter paper) or an artificial fiber material, with excrements.

The “transmitted light” specifically enables the visualization of individual ingredients in a receiving material for the optical measuring unit.

In addition, common test strip structures can be used, which consist, for example, of a carrier material, reagents, and an absorbent paper, etc.

The measurement can be performed with and without a supporting carrier material.

Plastic or cellulose can usually be used as a suitable carrier material for the indicator field.

A transmitted light measurement can be carried out in a structurally simple manner if the transmitted light measuring apparatus is arranged at least partially on both sides of the sample carrier.

An extremely precise detection can be achieved if the transmitted light measuring apparatus surrounds the sample carrier during the transmitted light measurement. This means that the sample carrier is surrounded by the sensor unit or the transmitted light measuring apparatus.

For this purpose, it is not only advantageous if the transmitted light measuring apparatus has an integral transport path, along which the sample carrier can be moved.

Among others, the integral transport path is characterized by the fact that it is protected from unwanted, critical ambient light.

At least sections of the transport path are preferably surrounded by the sensor unit or the transmitted light measuring apparatus, so that a very compact design, particularly of the sensor unit, can also be achieved.

Advantageously, the sensor unit has a housing, in which the transport path of the transmitted light measuring apparatus is at least partially arranged, resulting in a sample carrier to be transilluminated to be well protected from ambient light or the like during the transmitted light measurement.

The housing can be provided in a structurally very simple manner if it is at least partially configured by the guide part of the removal device or another suitable component of the device.

In addition, the sensor unit can be made even more compact if the transmitted light measuring apparatus has an illumination device on a first side of the integral transport path, and a detection device on a second side of the integral transport path which lies opposite the first side.

If, for example, the illumination device and the detection device are to be arranged as an alternative on a common side of the transport path, a mirror device, which, accordingly, can deflect light beams to the detection device, could be located on the other side opposite the illumination device.

The illumination device has at least one light source, while the detection device has at least one detection sensor surface.

In this case, it is advantageous for the herein described transmitted light measurement if the at least one light source lies opposite the at least one detection sensor surface.

However, with regard to another embodiment, it is also possible for at least one further light source to be arranged on the side of the detection device in order to be able to additionally illuminate the sample carrier.

In such case, illuminating means lying opposite the detection device can be used exclusively or also cumulatively to the further light sources, which are arranged on the side of the detection device, when operating the sensor unit. Or, alternatively to the further light sources, which are arranged on the side of the detection device, illuminating means lying opposite the detection device are used exclusively or cumulatively to perform the desired examinations.

Furthermore, it is advantageous if the transmitted light measuring apparatus has more than one light source, wherein a main beam path of at least one of the light sources is arranged to run at a right angle to a detection sensor surface, and at least one further main beam path of at least one further light source is arranged to run at a different angle to a detection sensor surface. This can further improve the quality of the analysis.

It goes without saying that the other devices and/or methods described herein can also be advantageously developed by means of such a transmitted light measuring apparatus or a sensor unit thus equipped.

A particularly preferred embodiment provides that the guide part comprises a sensor unit which is arranged on the side next to the guide path for guiding the movable arm element. As a result, the guidance of the movable arm element and the analysis of a sample can be separated with even more precision.

In relation to the narrow side of the guide path or arm element, “on the side” in the present case means right or left of the guide path or arm element.

However, the term “on the side” does not refer to a location on the upper side or the underside, and also not to an overlap with the broad side of the guide path or the arm element, and therefore also not above or below the guide path or the arm element.

If the clean region of the guide part, as seen looking in the extension direction of the movable arm element, is arranged axially in front of a sensor unit, it is structurally easily avoidable that the clean room is inadvertently contaminated by excrement when the used sample carrier is retracted because the used sample carrier only has to be moved as far as the sensor unit.

It is particularly expedient if the sensor unit is arranged at the free end of the guide part because this prevents the used sample carrier from having to be further retracted axially into the guide part in order to be able to analyze the adhering excrement.

If the sensor unit, as seen looking in the retraction direction of the movable arm element, is arranged axially in front of the clean region of the guide part, the used sample carrier does not even enter the clean region of the guide part for the analysis.

If the sensor unit, as seen looking in the retraction direction of the movable arm element, is arranged axially in front of a release unit for releasing a used sample carrier from the movable arm element, the used sample carrier can easily be released from the arm element after a completed analysis process by simply moving the arm element further in the retraction direction.

In addition, it is advantageous if, at the free end of the guide part, the sensor unit has a sensor region opening with a smaller opening height than a guide path opening of the guide path. Due to the smaller opening height of the sensor region opening, the sensor region is better protected against contamination from outside. As a result of the correspondingly larger opening height of the guide path opening, there is sufficient room height available for the movable arm element and the sample carrier temporarily held thereon, particularly for the relevant holder.

Cumulatively or alternatively, it is advantageous if the sensor region opening has a width that is greater than the width of the guide path. Due to the wider sensor region opening, the half of the sample carrier, which can be brought to overlap with the sensor unit, can be designed to be larger, particularly longer, than the half of the sample carrier, by means of which the latter is held on the holder of the movable arm element.

A further advantageous embodiment provides that the clean region of the guide part is wider than the contamination region of the guide part, particularly wider than the guide path. This can ensure that a new sample carrier on the guide part remains free of contamination as extensively as possible, so that the risk of accidental contamination of the sample carrier by an older excrement or the like can be reduced as much as possible.

In addition, it is expedient if the clean region of the guide part merges spatially into a sensor region of the guide part. As a result, the clean region and the sensor region can be placed spatially directly one behind the other in the axial movement direction, making the guide part very compact. In addition, a new sample carrier coming from the direction of the clean room can be guided over the sensor unit to the sampling in order to possibly already register the sample carrier before taking a sample or to check whether the new sample carrier is properly held on the movable arm element.

In order to ensure that the clean region of the guide part can at least partially overlap and protect the feeding device, it is advantageous if the clean region of the guide part is designed to be wider on its side facing away from the sensor region than on its side facing the sensor region.

In terms of design, the device can be kept very simple if both the contamination region and the clean region are arranged in a retaining bracket of the device. Advantageously, the guide part can thus serve directly as a retaining bracket. In particular, the transfer of a new sample carrier for sampling can thus be realized very easily.

The contamination region and the clean region are preferably arranged in a common retaining bracket.

If both the contamination region and the clean region are at least partially curved, these important regions can easily be integrated into a retaining bracket of the device.

A further embodiment, which is also advantageous without the other features of the invention, provides a sensor unit with a thermal sensor for detecting an excrement temperature, a body temperature, an excrement volume and/or an excrement volume flow.

Relevant parts of a toilet interior can advantageously be scanned by temperature sensors attached to the device, such as infrared sensors or the like, and thus further information can be obtained.

Due to the fact that corresponding thermal sensors are preferably arranged on the present device, they do not have to be permanently installed on the toilet. Instead, the thermal sensors are attached to the toilet in a mobile manner, so that they can be advantageously retrofitted to existing toilets.

A direct temperature measurement of the excrement can thus allow conclusions to be drawn about the body temperature of the person directly upon leaving the body or after contact with the toilet.

Suitable computing models and/or algorithms can be used to back-calculate or compensate for any temperature losses caused by outside air or toilet contact.

As a result, body temperature fluctuations can be recognized and, for example, conclusions about illnesses can be drawn.

A measurement of the temperature curve during the entire excretion phase also allows for the volume flow and thus also the dispensed quantity to be back-calculated.

In addition, the characteristic of emptying is revealed, which is also interesting information for some clinical pictures, for example, after kidney transplants or in case of urinary stones.

Advantageously, the same thermal sensors can also cumulatively trigger a measurement for determining a change in temperature within the toilet due to introduced excrements.

According to a fourth aspect of the invention, the problem addressed by the invention is solved by a device for the on-site analysis of excrements, comprising a housing, a removal device, by means of which a sample of excrement can be removed, in which the removal device comprises an arm element for a sample carrier, which is movable relative to the housing and which is guided at least partially in a guide part in such a way that it can be retracted and extended, a feeding device, by means of which a new sample carrier can be provided for the removal device, and an analysis device, by means of which the removed sample can be at least partially analyzed, wherein the device is characterized in that the removal device comprises a release unit with stripping means for releasing a used sample carrier from the arm element.

By means of such stripping means, a used sample carrier can be removed very reliably from the holder of the movable arm element, so that the device can be operated more reliably.

In particular, the risk that excrements in general, particularly excrements from another user, can inadvertently get deeper into the guide part, for example, in that a used sample carrier is not correctly removed from the holder of the movable arm element, can be completely excluded or at least significantly reduced. In any case, the present stripping means reduce the risk of falsified analysis results.

In this respect, a constant mobile monitoring using urine and/or stool samples can be performed easier and more reliable.

In order to increase the reliability of the present release unit, it is advantageous if the release unit is arranged on the guide part, particularly in a transition region at an axial height between a sensor region of the guide part and a clean region of the guide part. It can thus be achieved that a used sample carrier can still be moved into the sensor region by means of the movable arm element, but further in the direction toward the clean region, it is reliably detached from the holder of the movable arm element before the used sample carrier can continue to penetrate the clean room.

A particularly preferred embodiment provides that the release unit is designed such that the release unit can be passed by a holder arranged on the movable arm element for temporarily holding a sample carrier and/or activation means for activating the feeding device. This can ensure that a used sample carrier is reliably detached from the movable arm element, but the arm element, despite the holder and activation means arranged thereon, can still be retracted into the guide part in the retraction direction in order to be able to accept a new sample carrier in an operationally safe manner.

In this context, it is also advantageous if the release unit has a main part that can be penetrated by the movable arm element, wherein the main part at least partially surrounds a space with a room height that is many times greater than the arm element thickness. With such a room height, particularly the holder for the sample carrier and the aforementioned activation means or the like can easily pass the release unit.

It goes without saying that the room height can be selected particularly on the basis of the design of the holder or the activation means and also generally on the basis of the shape of the movable arm element.

It has already proven to be successful if the room height of the main part is more than twice, preferably more than ten times, the arm element thickness. In particular, a room height of more than ten times that of the arm element thickness, e.g., fifteen times, has proven to be sufficiently high, so that an interaction between the stripping means and the sample carrier as well as an unimpeded passing of the holder and the activation means is ensured.

It goes without saying that the stripping means suitable in the sense of the invention can be designed in a variety of ways.

For example, the stripping means can comprise brushes and/or bristles, by means of which a sample carrier is stripped from the holder of the movable arm element.

In practical tests, it has proven to be successful if the stripping means comprise at least one flexible finger element, and the movable arm element is guided past said finger element, preferably with contact, in a translational manner. For this purpose, the at least one flexible finger element can hold back the used sample carrier when the movable arm element is retracted further into the guide part in the axial retraction direction, so that the used sample carrier is ultimately released from the holder and subsequently falls, for example, into the toilet bowl and is thus disposed of.

For such purpose, the at least one flexible finger element can be in active contact with the movable arm element. It can also be positioned at a short distance above the movable arm element.

In such case, the at least one finger element extends from the main part of the release unit in the direction of the above-described guide path opening of the guide part and deviates in the direction toward the movable arm element, so that the free end of the finger element points to the movable arm element, either with contact between the free end of the finger element and the arm element or without contact.

For example, a new sample carrier coming from the feeding device can easily pass the release unit if the movable arm element is moved in the extension direction. However, if the movable arm element is retracted again and moved in the retracting direction, the sample carrier, which has been used in the meantime, gets caught on the free end of the finger element and is released from the holder by the further movement of the movable arm element.

In this respect, the release unit has a release tip which faces in the direction of the guide path opening of the guide part.

A further very advantageous embodiment provides that the flexible finger element is divided into two parts and has two flexible finger parts spaced apart by a gap. As a result, the sample carrier, as seen looking over the width of the movable arm element, can be easily caught and released by the flexible finger element, wherein the flexible finger element itself can nevertheless be designed in a very delicate manner.

If the two flexible finger parts are arranged at a distance from one another transversely to the axial movement direction or translational movement direction of the movable arm element, possible specifications of the movable arm element with a higher design can pass through the release unit between said two flexible finger parts.

The stripping means are preferably arranged on at least two sides of the movable arm element, so that a used sample carrier can also be penetrated from two sides, resulting in a still further increase of the operational safety of the release of the sample carrier from the holder of the movable arm element.

The stripping means are preferably arranged on the upper side and the underside of the arm element, so that the used sample carrier can be reliably caught and released from the holder, and thus stripped from the arm element.

If one flexible finger element with two flexible finger parts each is now placed on the upper side and the underside of the movable arm element, a used sample carrier can be caught with four stripping means and released from the holder.

In this last-mentioned configuration of the release unit, a total of at least four flexible finger parts form a release tip of the release unit, which faces in the direction of the guide path opening of the guide part.

According to a fifth aspect of the invention, the problem addressed by the invention is also solved by a device for the on-site analysis of excrements, comprising a housing, a removal device, by means of which a sample of excrement can be removed, in which the removal device comprises an arm element for a sample carrier, which is movable relative to the housing and which is guided at least partially in a guide part in such a way that it can be retracted and extended, a feeding device, by means of which a new sample carrier can be provided for the removal device, and an analysis device, by means of which the removed sample can be at least partially analyzed, wherein the device is characterized in that the feeding device can be activated by means of a movement of the movable arm element.

Since the feeding device can be activated, operated and controlled by means of the movement of the movable arm element, the design of the present device can be further simplified and thus also be operated more reliably.

In this respect, the present device is characterized in that the drive unit, by means of which the movable arm element is driven, is used to operate the feeding device.

In this respect, the removal device and the feeding device have the same drive.

In other words, the present invention, regardless of the other features of the invention, is also characterized in that the removal device and the feeding device have a single drive unit.

In addition, the use of the present device can be further improved if the device has an automatic micturition diary, by means of which measurement data can be stored.

Advantageously, ascertained information, such as urine parameters, time of the excrement delivery, duration of the excrement delivery, or the like, can be recorded directly in a digital micturition diary. The information can particularly be transmitted to digital terminals or medical systems for further processing.

Especially in connection with the features of the present holder, the problem addressed by the invention is also solved by a method for operating a device for the on-site analysis of excrements, in which a sample carrier is moved for sampling linearly relative to a sensor unit by means of an axially movable arm element, in which the sample carrier is held on a holder of the axially movable arm element, and in which a sample of excrement carried by the sample carrier is at least partially analyzed on the device, wherein the method is characterized in that a new sample carrier is fed to the holder transversely to the axial movement direction of the axially movable arm element, in that a feeding device is driven by means of the axial movement of the axially movable arm element in the axial movement direction.

By means of the method proposed herein, the device can be operated particularly efficiently because one and the same drive can be accessed for a plurality of functions.

In terms of design, the interaction between the movable arm element and the feeding device can be solved in various ways.

A structurally particularly simple embodiment provides that the feeding device has an activation lever which can be activated by means of a movement of the movable arm element. This activation lever can then be actuated by the movement of the movable arm element, preferably while the movable arm element is moving in the axial retraction direction.

Especially in this context, the problem addressed by the invention is also solved by a method for operating a device for the on-site analysis of excrements, in which a sample carrier is moved for sampling linearly relative to a sensor unit by means of an axially movable arm element, in which the sample carrier is held on a holder of the axially movable arm element, and in which a sample of excrement carried by the sample carrier is at least partially analyzed on the device, wherein the method is characterized in that a used sample carrier is released from the holder by means of the axial movement of the axially movable arm element in the axial movement direction.

By means of the method proposed herein, the device can be operated particularly efficiently because an additional drive for releasing a used sample carrier from the holder can be forgone.

An activation or actuation mechanism for the feeding device, which operates on the basis of the activation lever which can be activated by the movable arm element, can be kept structurally simple if the activation lever is rotatably mounted about an axis of rotation, wherein the axis of rotation for the activation lever is movable by means of a movement of the arm element. As a result, the feeding device can be moved translationally while the activation lever rotates about the axis of rotation.

In this respect, it is advantageous if the axis of rotation for the activation lever is movable transversely to the axial movement direction of the arm element.

In the present case, the structure of the feeding device can be realized very compactly if the feeding device has a movement curve, along which the activation lever can be rolled off as soon as the activation lever rotates about an axis of rotation of the activation lever. As a result, the activation lever can be guided on the axis of rotation and additionally by means of the movement curve.

If the feeding device has a movement curve, along which the activation lever can be rolled off when an axis of rotation of the activation lever is moved transversely to the axial movement direction of the arm element, the activation lever can be dragged in the direction of the movable arm element, while the activation lever is rotated about the axis of rotation by the movable arm element.

At this point, it should be noted that such a movement curve can be realized by different means for forcing a movement or a movement path, such as a sliding guide or the like. Furthermore, it is advantageous if the feeding device has a translationally movable transport carriage, by means of which a new sample carrier can be fed in the feed direction to a holder arranged on the movable arm element. Due to the translational movability of the transport carriage, the structure of the present feeding device can be simplified to a great extent.

If the transport carriage can be moved by means of a movement of the movable arm element, an additional drive for the transport carriage can be forgone.

The transport carriage can be dragged transversely to the axial movement direction of the movable arm element by means of the activation lever if the transport carriage has an axis of rotation for the activation lever.

In this respect, it is advantageous if the transport carriage can be moved by means of the activation lever transversely to the axial movement direction of the movable arm element and thus in the feed direction toward a holder for holding a new sample carrier. As a result, the design effort for the feeding device can fortunately be kept very low.

However, it is not only advantageous if the transport carriage can be moved translationally towards the movable arm element, but also if the transport carriage can be guided along a curved path in order to carry out a cutting movement perpendicularly to the feed direction for producing a new sample carrier. As a result, cutting means can readily be driven by means of the drive unit of the removal device or particularly of the movable arm element, which is extremely advantageous even without the other features of the present invention.

It goes without saying that said curved path can also be mechanically realized by various means for forcing a movement or a movement path. Once again, sliding guides or the like can be taken into consideration.

In this context, it is advantageous if the transport carriage comprises cutting means for cutting a new sample carrier from a sample carrier strip. An additional cutting device can thus be forgone.

Instead, the cutting means can already be configured by the transport carriage, or the transport carriage has corresponding seatings, in which cutting means can preferably be arranged in an exchangeable manner.

In this respect, the problem addressed by the invention is also solved by a method for operating a device for the on-site analysis of excrements, in which a sample carrier is moved for sampling linearly relative to a sensor unit by means of an axially movable arm element, in which the sample carrier is held on a holder of the axially movable arm element, and in which a sample of excrement carried by the sample carrier is at least partially analyzed on the device, wherein the method is characterized in that a new sample carrier is severed from a roll material, in that a cutting device is driven by means of the axial movement of the arm element in the axial movement direction.

By means of this method, the device can also be operated particularly efficiently because an additional drive for a cutting device can be forgone.

The roll material taken into consideration can be, for example, a filter paper or the like saturated with chemicals.

The roll material can be approximately 35 cm long and 0.5 cm wide.

In addition, a further preferred embodiment provides that the movable arm element comprises activation means for activating the feeding device. By means of the activation means on the arm element, the feeding device can be activated in a structurally simple manner by simply moving the arm element. In this respect, the drive of the movable arm element can be used to operate and control the feeding device.

In the sense of the invention, these activation means can be realized in different ways.

The activation means can be implemented in a very simple manner if the activation means comprise an elevation on the surface of the movable arm element.

Such an elevation can be embodied directly by the movable arm element, resulting in a further simplification of the present structure.

Cumulatively or alternatively, the elevation can be realized by a further component which is attached to the movable arm element in a suitable manner, for example, by a screw connection and/or an adhesive connection or the like.

In this case, the elevation can be provided, for example, by an additional pin part or the like on the movable arm element.

A further particularly preferred embodiment provides that the movable arm element comprises a holder which can be actuated by a movement of the movable arm element, wherein such a holder is advantageous even without the other features of the invention because even when considered in isolation, it already advantageously develops known devices of the type in question.

In any case, the movement of the movable arm element is used to actuate the holder, so that an additional drive for the holder can be omitted.

In this respect, the present holder is essentially based on a clamping mechanism, by means of which a sample carrier can be reversibly held or clamped on the movable arm element.

For this purpose, the holder can be attached, particularly placed or clipped onto, the movable arm element, or the holder is preferably embodied directly by means of the movable arm element.

For example, plastic, metal, or possibly also another material, or a combination thereof, can be taken into consideration for the material of the holder.

In any case, it is expedient if the holder comprises at least two clamping elements, wherein at least one clamping element can be actively actuated.

Furthermore, it is advantageous if the device has a holder actuation member for actuating a holder arranged on the movable arm element for temporarily holding the sample carrier, wherein the holder actuation member comprises a ramp element.

In this case, the holder can be pulled over said ramp element by means of the movement of the movable arm element, resulting in the elastic deflection of at least one clamping element, in order to clamp a new sample carrier between the at least two clamping elements and thus hold it on the movable arm element.

Especially in connection with the features of the present holder, the problem addressed by the invention is also solved by a method for operating a device for the on-site analysis of excrements, in which a sample carrier is moved for sampling linearly relative to a sensor unit by means of an axially movable arm element, in which the sample carrier is held on a holder of the axially movable arm element, and in which a sample of excrement carried by the sample carrier is at least partially analyzed on the device, wherein the method is characterized in that the holder is opened or closed by means of the axial movement of the axially movable arm element in the axial movement direction.

By means of the method proposed herein, the device can be operated particularly efficiently because an additional drive for actuating the holder can be forgone.

The problem addressed by the invention is furthermore also solved by a method for operating a device for the on-site analysis of excrements, in which a sample carrier is moved for sampling linearly relative to a sensor unit by means of an axially movable arm element, in which the sample carrier is held on a holder of the axially movable arm element, and in which a sample of excrement carried by the sample carrier is at least partially analyzed on the device, wherein the method is characterized in that a sample carrier provided with excrement is moved between an illumination device and a detection device and transilluminated for a transmitted light measurement by means of the illumination device.

In this case, a particularly high analysis quality can be achieved if the sample carrier provided with excrement is at rest during the transmitted light measurement.

However, the method can be carried out more quickly if the sample carrier provided with excrement is moved during the transmitted light measurement.

With regard to additional advantageous method steps, reference is made to the description, particularly to the device with the transmitted light measuring apparatus, in order to avoid repetitions in the present case.

A further method variation provides that a moistening of the sample carrier with excrement, particularly with urine, is supported by means of additional movements, particularly in the axial extension and/or retraction direction, of the axially movable arm element. As a result, the duration of a sampling can be shortened considerably.

With regard to another method variation, it is also advantageous if the duration of a sampling is between 10 s and 1 s, preferably between 2 s and 3 s. With such a short period of time for sampling, the use of the device can be temporally limited to a great extent. In addition, this reduces the risk of undesired contamination of the sample by external influences because the faster the removed sample can be fed to the sensor unit within the guide part, the lower the risk of undesired external influences.

In addition, it is advantageous if excess excrement, particularly excess urine, is removed from the movable arm element, the sample carrier and/or the holder by a vibration of said axially movable arm element. By removing excess excrement, the risk of contamination of the device, particularly of the guide part, can additionally be reduced favorably because ideally only a small amount of excrement adheres to the sample carrier for the sensor examination.

With regard to the methods described herein, it must also be noted at this point that the individual methods can additionally be supplemented by device features in order to further specify method sequences.

The design of the present device can be made even more compact if the guide part has an articulated connection, by means of which the guide part is arranged on the housing in an articulated manner. By means of this articulated connection, the guide part can be folded onto the housing when not in use.

In this respect, the problem addressed by the present invention is, according to a further aspect of the invention, also solved by a retaining bracket for a device for the on-site analysis of excrements for attachment to a toilet, a urinal or the like, comprising a guide path, along which a movable arm element of the retaining bracket is guided in a retractable and extendable manner, wherein the retaining bracket has a contamination region with the guide path for guiding the movable arm element and also a clean region, in which a new sample carrier can be arranged at least partially next to the guide path.

With a retaining bracket thus structured, the risk of unintentional contamination of a new sample carrier by old excrements is significantly reduced.

The simplifications of the device proposed herein not only result in a higher reliability of the device, but also reduce weight due to the associated reduction of components, and effect an extremely efficient operation of the device.

In particular, the risk that a new sample carrier is not or not completely inserted into a holder for holding the sample carrier can be completely eliminated or at least significantly reduced.

In this respect, a constant mobile monitoring using urine and/or stool samples is even easier and more reliable.

It goes without saying that the features of the solutions described above or in the claims can also be combined, if appropriate, in order to be able to implement the advantages and effects achievable in the present case in a correspondingly cumulative manner

At this point, it must also be mentioned that, in the context of the present patent application, the term “in particular” or “particularly” always refers to the introduction of an optional, preferred feature. The term is not meant to indicate “i.e.” and/or “namely.”

Furthermore, it must be pointed out that in the context of the present patent application, indefinite articles and indefinite numerical data, such as “one . . . ,” “two . . . ,” etc., generally refer to minimum specifications, i.e., to “at least one . . . ,” “at least two . . . ,” etc., unless it is clear from the context or the specific text of a particular passage that they refer only to “exactly one . . . ,” “exactly two . . . ,” etc.

In addition, further features, effects and advantages of the present invention are described by means of the attached drawings and the following description, in which a device for the on-site analysis of excrements is depicted and described by way of example.

Components which at least essentially correspond in terms of their function in the individual drawings can be denoted with the same reference signs, wherein the components are not necessarily denoted with reference signs and described in all the drawings.

It must be pointed out that the drawings shown are depictions which illustrate the fundamental structure and the fundamental mode of operation.

The drawings show in:

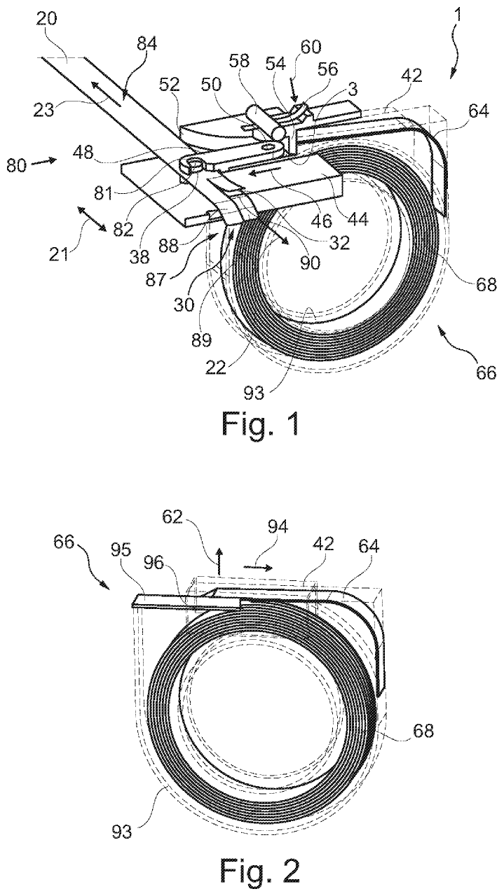

FIG. 1 schematically a first perspective view of a partially depicted device for the on-site analysis of excrements in a starting position;

FIG. 2 schematically a second perspective view of the device shown in FIG. 1 with a magazine comprising a material roll for new sample carriers;

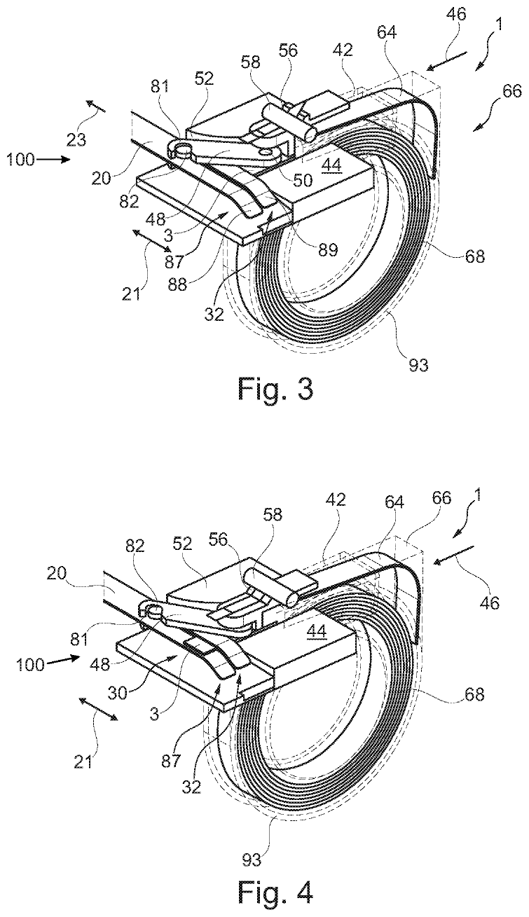

FIG. 3 schematically a third perspective view of the device shown in FIGS. 1 and 2 in a feeding position, in which a sample carrier is fed to the holder of the movable arm element;

FIG. 4 schematically a fourth perspective view of the device shown in FIGS. 1 to 3 in a cutting position, in which the sample carrier partially fed to the holder is severed from the material roll;

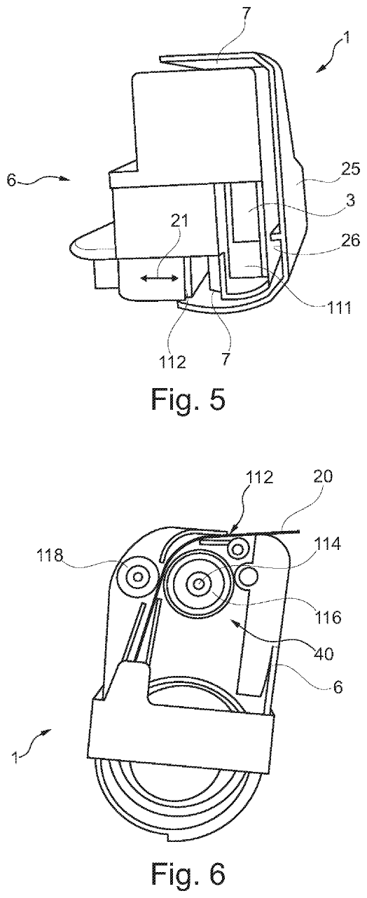

FIG. 5 schematically a further view of the device shown in FIGS. 1 to 4 with a housing and the guide part folded thereon;

FIG. 6 schematically a view of the removal device of the device shown in FIGS. 1 to 5 with the partially rolled up movable arm element and its drive;

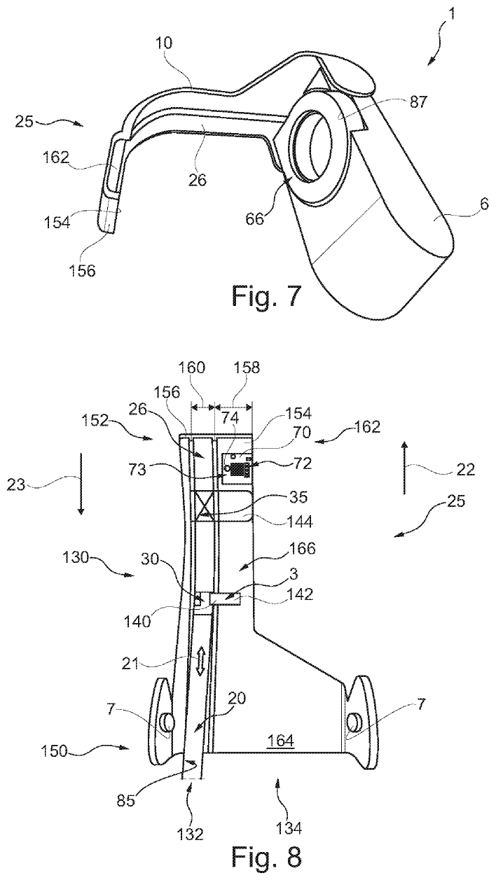

FIG. 7 schematically a further perspective view of the device shown in FIGS. 1 to 6 with folded guide part;

FIG. 8 schematically a bottom view of the guide part of the device shown in FIGS. 1 to 7 with a view of the contamination region, the clean region, and the sensor region of the guide part;

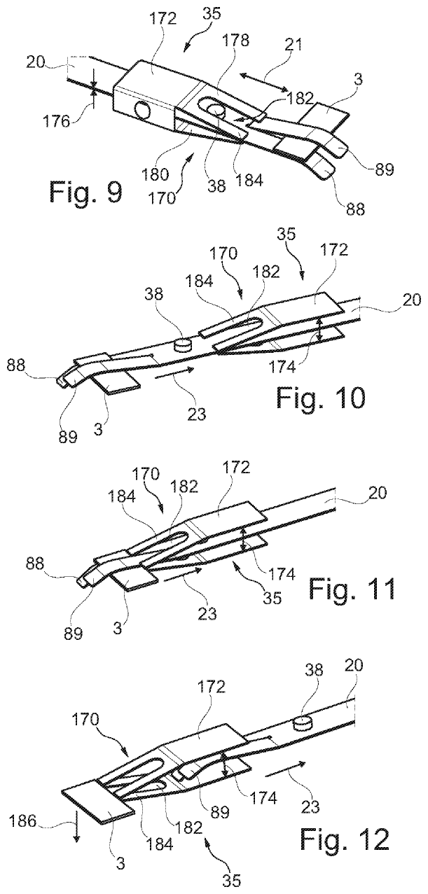

FIG. 9 schematically a first perspective detail view of the release unit of the device shown in FIGS. 1 to 8;

FIG. 10 schematically a second perspective detail view of the release unit of the device shown in FIGS. 1 to 9;

FIG. 11 schematically a third perspective detail view of the release unit of the device shown in FIGS. 1 to 10;

FIG. 12 schematically a fourth perspective detail view of the release unit of the device shown in FIGS. 1 to 11;

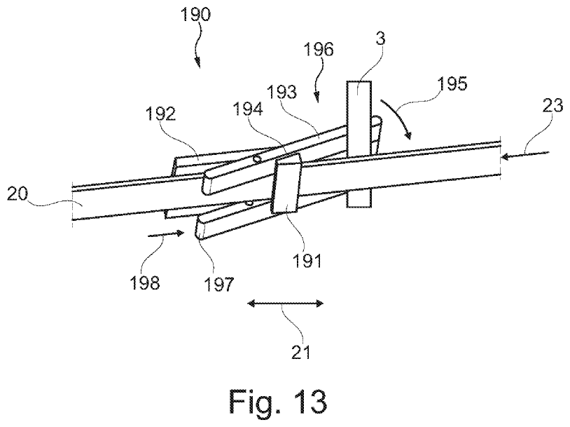

FIG. 13 schematically a bottom view of an alternative holder for temporarily holding a sample carrier on a movable arm element;

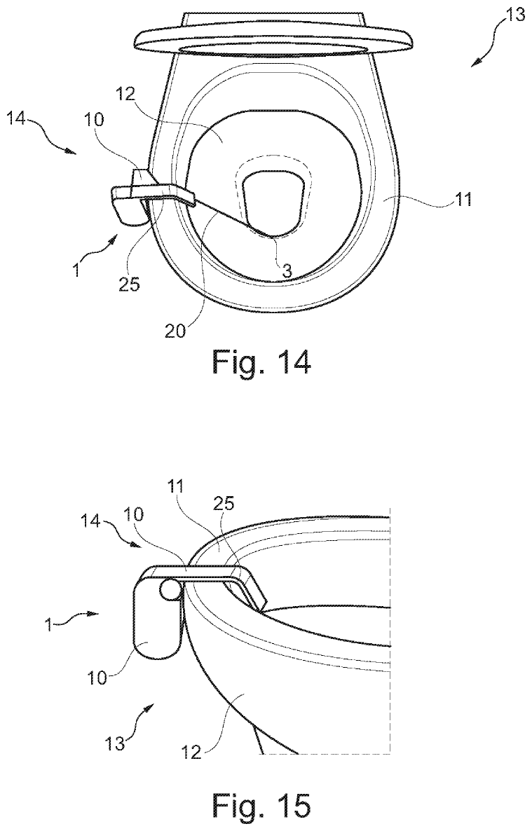

FIG. 14 schematically a view of an arrangement consisting of the device shown in FIGS. 1 to 12 and a toilet;

FIG. 15 schematically a detail view of the arrangement shown in FIG. 14;

FIG. 16 schematically a view of an advantageous sensor unit with a transmitted light measuring apparatus;

FIG. 17 schematically a further view of the sensor unit from FIG. 16;

FIG. 18 schematically a view of an alternative sensor unit with a transmitted light measuring apparatus;

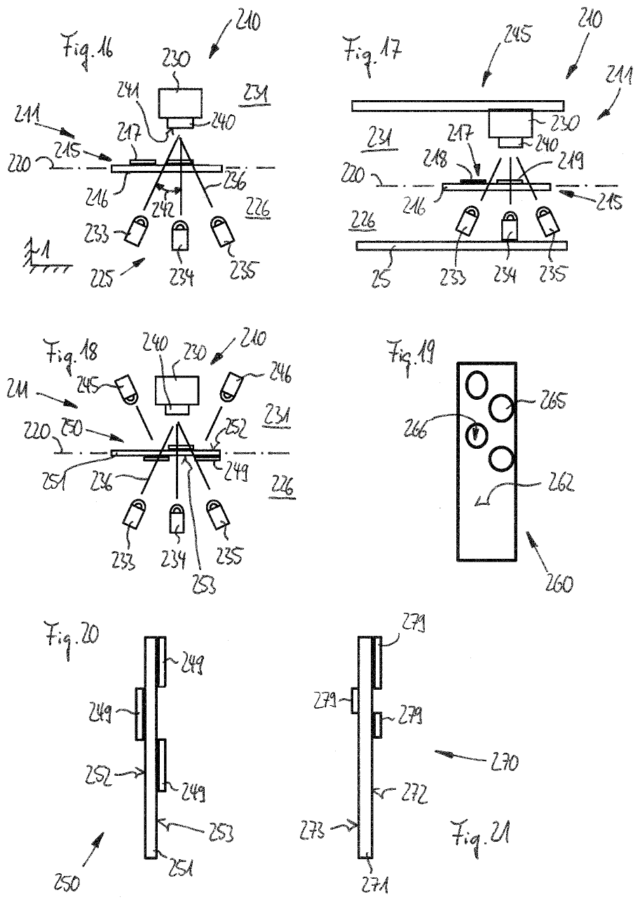

FIG. 19 schematically a top view of a sample carrier with occasional round and oval sample analysis fields;

FIG. 20 schematically a side view of a sample carrier with occasional sample analysis fields arranged on both sides; and

FIG. 21 schematically a side view of a further sample carrier with occasional sample analysis fields of different sizes arranged on both sides.

The device 1 for the on-site analysis of excrements, which is shown fundamentally in FIGS. 1 to 15 with regard to its structure and mode of operation, essentially consists of a removal device 2, by means of which a sample of excrement can be removed by a sample carrier 3, a feeding device 4, by means of which a new sample carrier 3 can be provided for the removal device 2, and an analysis device 5, by means of which a removed sample can be at least partially analyzed directly with the device 1.

The device 1 comprises a housing 6, to which a retaining bracket 10 is fastened to be folded up or down by means of a articulated connection 7.

By means of said retaining bracket 10, the device 1 can be attached, for example, to an edge 11 of a toilet bowl 12 of a toilet 13, as is shown with regard to the corresponding arrangement 14 according to the depictions in FIGS. 14 and 15.

The removal device 2 essentially comprises an arm element 20, which is movable relative to the housing 6 of the device 1, for moving the sample carrier 3 in an axial movement direction 21, more precisely, in an axial extension direction 22 or in an axial retraction direction 23, a guide part 25 with a guide path 26 for precisely guiding the movable arm element 20, a holder 30 for holding a sample carrier 3 on the movable arm element 20, an opening and closing unit 32 for opening and closing the holder 30, a release unit 35 for releasing a used sample carrier 3 from the holder 30, as well as activation means 38 for activating the feeding device 4, and a drive unit 40 for axially driving the movable arm element 20.

The feeding device 4 essentially comprises a translationally movable transport carriage 42, which is movable on a guide path part 44 in a feed direction 46 running transversely to the axial movement direction 21, an activation lever 48 which is rotatably mounted on an axis of rotation 50 of the transport carriage 42, a movement curve 52, on which the activation lever 48 can also be rolled off, cutting means 54 of a cutting direction (not depicted herein) for producing individual sample carriers 3, and an additional curved path 56 which slides along a guide pin 58 of the housing 6 in order to guide particularly the cutting means 54 in the cutting direction 60 transversely to the feed direction 46 and against a spring force 62 of a spring element 64, and finally a refillable magazine 66 for storing roll material 68, from which new sample carriers 3 can be cut by means of the cutting means 54.

At this point, it must also be noted that instead of roll material 68 with a suitably designed magazine (not explicitly depicted herein), other storage configurations, for example, pre-produced strip elements or the like, can also be provided as sample carriers. In this way, the cutting device 54 can possibly be further simplified or can be omitted entirely.

It goes without saying that the depicted movement curve 52 and the additional curved path 56 are only a first option of many constructive options for deflecting the activation lever 48 or the transport carriage 42 or the cutting device in a desired manner. Instead, it is also possible to use other sliding guides, in which a sliding block is guided in a sliding groove, etc.

In addition to an analysis unit 70, the analysis device 5 comprises a sensor unit 72 with a multiplicity of different sensors 73 (herein denoted only by way of example) and a data transmission unit 74 for transmitting analysis data or information to almost any receiver device, e.g., a smartphone, on which a corresponding application for visualizing and/or further analyzing the data or information is running.

For this purpose, however, the analysis unit 70 can also be situated at another location on the device, for example, in the housing 6.

For example, the analysis unit 70 comprises a microcontroller (not depicted) or the like for analysis purposes.

In this case, the analysis unit 70 and the sensor unit 72 can be in active contact with one another in a wired or wireless manner. This also applies to the data transmission unit 74 or the like already mentioned above

According to the depiction in FIG. 1, the device 1 is in a starting position 80, in which the movable arm element 20 is retracted into the guide part 25 such that the activation means 38 are already located in a groove 81 of the activation lever 48, wherein, however, the activation lever 48 has not yet been rotated about the axis of rotation 50 by the activation means 38, or if it has been rotated, then only to a negligible extent.

In this embodiment, the activation means 38 are designed, by way of example, as elevations 82 on the upper side of the movable arm element 20.

In this embodiment, said elevation 82 is designed as a pin element (not explicitly denoted again).

It goes without saying that, with a corresponding redesign of the device 1, this elevation 82 or the non-denoted pin element can also be configured on the underside 85 of the movable arm element 20.

In any case, the activation lever 48 is still in an inactivated state and is essentially still aligned perpendicularly to the axial movement direction 21.

By means of the transport carriage 42, the roll material 68 is already repositioned below the cutting means 54.

The present cutting means 54 can be designed differently, for example, as a cutting knife, ripping knife, or punching knife, so that the term cutting means 54 not only refers to cutting, but also to punching, tearing, or other cutting methods.

The transport carriage 42 is pressed upward, i.e., in the opposite direction of the actual cutting direction 60, by means of a spring force 62 of the leaf spring element 64 which is arranged in the magazine housing 87.

In this case, the cutting means 54 are preferably exchangeable with the magazine housing 87 as soon as the roll material 68 has been used up.

According to the depiction in FIG. 2 which essentially shows the magazine 66 of the device 1, it is clarified once more that the transport carriage 42 and the leaf spring element 64 interact with one another such that the transport carriage 42 is not only moved in the upward direction by the spring forces 62 against the cutting direction 60, but also in the rearward direction 88 away from the movable arm element 20.

Below the transport carriage 42, the magazine 66 is also equipped with a closure part 89, which can essentially follow the movement of the transport carriage 42 and, when inactive, can close the magazine housing 87 at the feed opening 90, from which the roll material 68 is conveyed. In this way, the roll material 68 can also be well protected, for example, from atmospheric moisture.

The holder 30 for holding the respective sample carrier 3 is arranged on the front, free end 87 of the movable arm element 20, wherein the holder 30 has two clamping elements 88 and 89 in this embodiment.

At least the second clamping element 89 can be elastically deflected by a holder actuation member 90, so that the holder 30 can be opened by the holder actuation member 90; as a result, a new sample carrier 3 can then be inserted into the holder 30 by means of the feeding device 4.

At this point, it must also be noted that, according to the depiction in FIG. 1, the second clamping element 89 is shown partially cut open in order to visualize the ramp element 91 hidden under the second clamping element 89.

Furthermore, in this embodiment, the holder actuation member 90 is designed as a ramp element 91 (cf. particularly FIG. 4), with which the second clamping element 89 can collide, so that it is deflected upward.

According to the depiction in FIG. 3, the device 1 is already shown in a more advanced feeding position 100, in which the movable arm element 20 is visualized further in the axial retraction direction 23, so that the activation lever 48 has already been rotated further about the axis of rotation 50, wherein the activation lever 48 is simultaneously supported by the movement curve 52.

As a result, the transport carriage 42 is moved towards the movable arm element 20 in the feed direction 46, and the sample carrier 3 can thus now be inserted further into the more open holder 30.

The movement of the transport carriage 42 in the feed direction 46 towards the movable arm element 20 can be easily recognized by the fact that the curved path 56 has already moved further relative to the guide pin 58.

Due to the axial movement of the movable arm element 20 in the axial retraction direction 23, the second clamping element 89 of the holder 30 is deflected upward because its curved end (not denoted again) increasingly collides with the ramp element 91.

According to the depiction in FIG. 4, the device 1 is shown in a cutting position 110, in which the movable arm element 20 is moved still further in the axial retraction direction 23, so that the transport carriage 42 is moved even further toward the movable arm element 20, and the curved path 56 is again guided further under the guide pin 58, so that the transport carriage 42 and the cutting means 54 attached thereto have been moved downward in the cutting direction 60.

In this respect, a new sample carrier 3 is now severed from the roll material 68 located in the magazine 66.

It can also be clearly seen that the second clamping element 89 has now completely collided with the ramp element 91, and the holder 30 is thus completely open, and the new sample carrier 3 can now be passed on from the feeding device 4 to the holder 30 in an unimpeded manner.

When the new sample carrier 3 is finally correctly positioned in the holder 30, the movable arm element 20 is moved in the axial extension direction 22, and the second clamping element 89 is therefore again moved away from the ramp element 91, so that, as a consequence, the holder 30 closes and the new sample carrier 3 is clamped firmly and operationally safe in the holder 30, although this is once again not explicitly shown.

According to the depiction in FIG. 5, the device 1 is shown with its housing 6 and the retaining bracket 10 arranged thereon, wherein a transfer region 111 is shown between the feeding device 4 and the removal device 2 with a sample carrier 3 positioned there by way of example. In this case, the movable arm element 20 is not shown again, wherein said movable arm element 20 normally protrudes from the outlet opening 112.

According to the depiction in FIG. 6, this is shown in connection with the drive unit 40 of the removal device 2, wherein the movable arm element 20 is shown rolled up and already guided out of the outlet opening 112.

Representative of the drive unit 40, only the drive shaft 114 with the drive roller 116 arranged thereon and the pressure roller 118 are shown, wherein the movable arm element 20 is guided between the drive roller 116 and the pressure roller 118.

Otherwise, the movable arm element 20 is largely rolled up in the housing 6.

According to the depiction in FIG. 7, the device 1 is shown with the guide part 25 folded down, so that the device 1, as shown in FIGS. 14 and 15, can be attached to the edge 11 of the toilet bowl 12.

In the present case, the guide part 25 is advantageously designed as a retaining bracket 10, so that the device 1 can be constructed in a very compact manner.

According to the depiction in FIG. 8, the guide part 25 is shown in more detail from its underside 130, wherein it can be clearly seen that the guide part 25 extends into a contamination region 132, in which the guide path 26 extends in the axial movement direction 21, and also divides a clean region 134, in which a new sample carrier 3 can be guided at least partially next to the guide path 26, and particularly the sensor unit 72 is arranged.

In this case, the clean region 134 is arranged on the side next to the contamination region 132, wherein, between the contamination region 132 and the clean region 134, a separation 136 is provided which extends with a rigid partition 138 in the axial movement direction 21 in the longitudinal extension of the guide part 25.

In this case, the partition 138 is arranged and, above all, configured such that it can be bridged by a sample carrier 3 clamped onto the holder 30.

In other words, this means that the new sample carrier 3, held on the holder 30, with its clamping side 140 is located in the contamination region 132, while the sample carrier 3 with its sensor side 142 is located in the clean region 134.

This can ensure that the sensor side 142 of a new sample carrier 3 is not already being contaminated with excrement on its path in the axial extension direction 22 because a previously used sample carrier 3 can only reach the axial height of the sensor unit 72 and maximally the axial height of the release unit 35 or a transition region 144 arranged in a radially adjacent manner.

In this embodiment, the guide part 25 is open on its underside 130, so that particularly the contamination region 132 and the clean region 134 can always be cleaned or disinfected well.

In this case, the clean region 134, as seen looking in the axial retraction direction 23, is arranged axially behind the release unit 35.

In this respect, the clean region 134, as seen looking in the axial extension direction 22, is arranged axially in front of the sensor unit 72.

The guide path 26 extends from the rear end 150 of the guide part 25 to the front end 152 of the guide part 25.

The clean region 134 extends essentially from the rear end 150 of the guide part 20 to the transition region 144, wherein the sensor unit 72 adjoins said transition region 144 at the front, free end 152.

At the front, free end 152, the sensor unit 72 has a sensor region opening 154 with a smaller opening height (not explicitly denoted) than a guide path opening 156 of the guide path 26.

In this case, the sensor region opening 154 has a width 158 which is greater than the width 160 of the guide path 26.

Furthermore, the clean region 134 is configured wider on its side 164 facing away from the sensor region 162 than on its side 166 facing the sensor region 162.

According to the depictions in FIGS. 9 to 12, the release unit 35 is illustrated again in more detail in combination with the holder 30 of the axially movable arm element.

As can already be seen in FIG. 8, the release unit 35 is arranged in the transition region 144 at an axial height between the sensor region 162 and the clean region 134.

The release unit 35 has stripping means 170, which are designed such that they only have a stripping or releasing effect on the sample carrier 3 in the axial retraction direction 23 (see particularly FIGS. 10 to 12), so that only a used sample carrier 3 is released from the holder 30 when the axially movable arm element 20 is moved through the release unit 35 in the axial retraction direction 23.

For this purpose, the release unit 35 has a main part 172 that can be penetrated by the axially movable arm element 20.

In this case, the main part 172 has a room height 174 which in this embodiment is more than 10 mm.

In any case, the room height 174 is many times greater than the arm element thickness 176.

In this embodiment, the stripping means 170 of the release unit 35 are designed as a first finger element 178 on the upper side 84 of the axially movable arm element 20 and as a lower flexible finger element 180 on the underside 85 of the axially movable arm element 20.

Both flexible finger elements 178 and 180 are two-part finger elements.

In other words, each of the flexible finger elements 178, 180 has two flexible finger parts 184 spaced apart from one another by a gap 182 (denoted only by way of example).

For this purpose, the gap 182 is selected such that the activation means 38 can easily pass through it, so that the activation means 38 do not influence the task of the release unit 35, but only serve to activate the feeding device 4 already described in detail above.

In addition, it is advantageous that the flexible finger parts 184 can adapt to, or flexibly follow, the existing contours of the axially movable arm element 20, so that the actual holder 30 configured on the axially movable arm element 20 can pass said release unit 35 without any problems.

In this respect, the release unit 35, in the sense of the invention, only interacts with a used sample carrier 3, so that it is released from the holder 30 of the axially movable arm element 20 when the axially movable arm element 20 with the holder 30 is moved through the release unit 35 in the axial retraction direction 23.

Only then is the used sample carrier 3 released from the holder 30 by means of the flexible finger parts 184, and the used sample carrier 3 falls into the toilet bowl 12 (see FIGS. 14 and 15) according to the disposal direction 186 (see FIG. 12), so that it can be easily disposed of via the toilet 13.

The alternative holder 190 shown in FIG. 13 has a fastener 191 with a fastening body 192, by means of which the holder 190 can be fixed on the movable arm element 20. For this purpose, it is sufficient to simply slide the holder 190 onto the movable arm element 20 with a mounting force, wherein the mounting force is substantially greater than all other working forces acting on the holder 190 during normal operation of the device 1. As a result, the holder 190 sits operationally safe on the movable arm element 20.

The holder 190 has a clamping element 193 which is rotatably mounted on the fastening body 192 by means of a spring-pretensioned axis of rotation 194.

In this case, the holder 190 functions essentially similarly to a spring-pretensioned folding mechanism.

Basically, the clamping element 193 is moved in the clamping direction 195 by the spring forces of the spring-pretensioned axis of rotation 194, so that a sample carrier 3 is clamped operationally safe between the movable arm element 20 and the clamping element 193.

The holder 190 is opened by moving the movable arm element 20 in the retraction direction 23 and pressing the end 197 of the clamping element 193 facing away from the clamping side 196 against a resistance (not depicted), resulting in an opening force 198 acting on said end 197.

Said resistance is normally located on the feeding device 4 because this is where the holder 190 is also supposed to be opened for receiving a new sample carrier 3.

The resistance can be realized by a mechanical forced guidance or other mechanical elements, which can be arranged along the movable arm element 20 preferably in an adjustable manner. In this way, the position, at which the holder 190 is finally opened in order to receive a new sample carrier 3, or lets go of a used sample carrier 3 and releases it from the holder 190, can be clearly determined.

According to the depiction in FIG. 14, the present device 1 is suspended from the edge 11 of the toilet bowl 12. For this purpose, the axially movable arm element 20 has already been extended, particularly from the guide part 25, into the toilet bowl 12, wherein a new sample carrier 3 is placed inside the toilet bowl 12 such that the sample carrier 3 is well placed on the toilet 13 for receiving particularly urine.

According to the further depiction in FIG. 15, it is shown again in somewhat more detail, how the device 1 is placed on the toilet bowl 12 by means of the retaining bracket 10.

According to the depictions in FIGS. 16 and 17, another sensor unit 210 with a transmitted light apparatus 211 is also shown, wherein particularly the device 1 described above can also be equipped cumulatively or alternatively with said other sensor unit 210.

By means of said other sensor unit 210, it is possible to not only illuminate a sample carrier 215 from one side, for example, in the sense of reflection photometry, but to also completely or partially transilluminate a correspondingly designed sample carrier 215.

For this purpose, the sample carrier 215 has a translucent main part 216 and translucent sample analysis fields 217 (denoted only by way of example), wherein said translucent sample analysis fields 217 can also be equipped with different indicators 218 and 219 for different analyses (only denoted by way of example, cf. FIGS. 17 and 21).

The transmitted light measuring apparatus 211 is arranged at least partially on both sides of the sample carrier 215.

More precisely, the transmitted light measuring apparatus 211 is at least partially arranged on both sides of a transport path 220, wherein the transport path 220 is an integral component of the transmitted light measuring apparatus 211, along which the sample carrier 215 can be moved.

In particular, the transmitted light apparatus 211 in this embodiment has an illumination device 225 on a first side 226 of the integral transport path 220, and a detection device 230 on a second side 231 of the integral transport path 220 which lies opposite the first side 226.

In this case, the illumination device 225 has three light sources 233, 234, and 235 with respective main beam paths 236 (denoted only by way of example), wherein the light sources 233, 234, and 235 are herein designed as LEDs.

The detection device 230 is characterized by at least one detection sensor surface 240, which in this embodiment is arranged with its detection side 241 facing the light sources 233, 234, and 235.

The transmitted light measuring apparatus 211 thus has more than one light source 233, 234, 235, wherein the main beam path 236 of the light sources 234 is arranged to run at a right angle to the detection sensor surface 240, and the main beam paths 236 of the further light sources 233 and 235 are arranged to run at a different angle 242 to the detection sensor surface 240.

The other sensor unit 210 can be equipped with a simple housing 245 consisting at least partially of the guide part 25 of the device, as can be seen in FIG. 17.

According to the depiction in FIG. 18, the sensor unit 210 shown in FIGS. 16 and 17 is equipped with the transmitted light measuring apparatus 211 and also has two additional light sources 245 and 246, which are arranged on the same side of the integral transport path 220 as the detection device 230, namely the second side 231.

By means of the two additional light sources 245 and 246, sample analysis fields 249 of the alternative sample carrier 250 introduced into the sensor unit 210 can either be additionally transilluminated or illuminated in a reflective manner.

On the alternative sample carrier 250, the sample analysis fields 249 are arranged on both sides of its main part 251 (denoted only by way of example), namely a single sample analysis field 249 on its front side 252, and two sample analysis fields 249 on its rear side 253, so that, at an identical sample carrier size, more sample analysis fields 249 can be accommodated on the alternative sample carrier 250.

According to the depiction in FIG. 19, another sample carrier 260 is shown, on the front side 262 of which round sample analysis fields 265 of different sizes (only denoted by way of example) with different indicator materials 266 (only denoted by way of example) are placed.

FIG. 20 shows a side view of the sample carrier 250 from FIG. 18.

FIG. 21 shows a further alternative sample carrier 270. The further alternative sample carrier 270 has a main part 271, on the front 272 of which two sample analysis fields 279 of different sizes are placed, and on the rear 273 of which a single sample analysis field 279 is placed.

At this point, it must be explicitly pointed out that features of the solutions described above or in the claims and/or the drawings can also be combined, if appropriate, in order to be able to implement or achieve the described features, effects, and advantages in a correspondingly cumulative manner.

It goes without saying that the embodiment described above is merely a first embodiment of the device according to the invention. In this respect, the design of the invention is not limited to said embodiment.

All of the features disclosed in the application documents are claimed to be essential to the invention, provided that, individually or in combination with one another, they are novel over the prior art.

### LIST OF REFERENCE SIGNS USED

1 Device for the on-site analysis of excrements
2 Removal device
3 Sample carrier (new or used)
4 Feeding device
5 Analysis device
6 Housing
7 Articulated connection
10 Retaining bracket
11 Edge
12 Toilet bowl
13 Toilet
14 Arrangement
20 axially movable arm element
21 axial movement direction
22 axial extension direction
23 axial retraction direction
25 Guide part
26 Guide path
30 Holder
32 Opening and closing unit
35 Release unit
38 Activation means
40 Drive unit
42 Transport carriage
44 Guide path part
46 Feed direction
48 Activation lever
50 Axis of rotation
52 Movement curve
54 Cutting means
56 Curved path
58 Guide path
60 Cutting direction
62 Spring forces
64 Leaf spring element
66 Magazine
68 Roll material
70 Analysis unit
72 Sensor unit
73 Sensors (identical or different)
74 Data transmission unit
80 Starting position
81 Groove
82 Elevation
84 Upper side
85 Underside
87 front, free end
88 first clamping element
89 second clamping element
90 Holder actuation member
91 Ramp element
93 Magazine housing
94 rearward direction
95 Closure part
96 Feed opening
100 Feeding position
110 Cutting position
111 Transfer region
112 Outlet opening
114 Drive shaft
116 Drive roller
118 Pressure roller
130 Underside
132 Contamination region
134 Clean region
136 Separation
138 Partition
140 Clamping side
142 Sensor side
144 Transition region
150 rear end
152 front, free end
154 Sensor region opening
156 Guide path opening
158 Width
160 Width
162 Sensor region
164 the side facing away
166 the side facing
170 Stripping means
172 Main part
174 Room height
176 Arm element thickness
178 upper finger element
180 lower finger element
182 Gap
184 flexible finger parts
186 Disposal direction
190 alternative holder
191 Fastener
192 Fastening body
193 Clamping element
194 spring-pretensioned axis of rotation
195 Clamping direction
196 Clamping side
197 the end facing away
198 Opening force
210 other sensor unit
211 Transmitted light measuring apparatus
215 Sample carrier
216 Main part
217 Sample analysis fields
218 a first indicator
219 a further indicator
220 Transport path
225 Illumination device
226 first side
230 Detection device
231 second side
233 first light source
234 second light source
235 third light source
236 Main beam paths
240 Detection sensor surface
241 Detection side
242 Angle
245 first additional light source
246 second additional light source
249 Sample analysis fields
250 alternative sample carrier
251 Main part
252 Front side
253 Rear side
260 other sample carrier
262 Front side
265 round sample analysis fields
266 different indicator materials
270 further alternative sample carrier
271 Main part
272 Front side
273 Rear side
279 Sample analysis fields

1-63. (canceled)

64. Device for the on-site analysis of excrements, comprising a housing, a removal device, by means of which a sample of excrement can be removed, in which the removal device comprises an arm element for a sample carrier, which is movable relative to the housing in the axial movement direction, said arm element being guided at least partially in a guide part in such a way that it can be retracted and extended, a feeding device by means of which a new sample carrier can be provided for the removal device, and an analysis device, by means of which the removed sample can be at least partially analyzed, characterized in that the guide part comprises a contamination region with a guide path for guiding the movable arm element, and also a clean region, in which a new sample carrier can be arranged partially next to the guide path.

65. Device for the on-site analysis of excrements, comprising a housing, a removal device, by means of which a sample of excrement can be removed, in which the removal device comprises an arm element for a sample carrier, which is movable relative to the housing in the axial movement direction, said arm element being guided at least partially in a guide part in such a way that it can be retracted and extended, and an analysis device, by means of which the removed sample can be at least partially analyzed, characterized by a sensor unit having a transmitted light measuring apparatus for transilluminating the sample carrier.

66. Device according to claim 65, characterized in that the transmitted light measuring apparatus is at least partially arranged on both sides of the sample carrier.

67. Device according to claim 65, characterized in that the transmitted light measuring apparatus has an integral transport path, along which the sample carrier can be moved.

68. Device according to claim 67, characterized in that the transmitted light measuring apparatus has an illumination device on a first side of the integral transport path, and a detection device on a second side of the integral transport path which lies opposite the first side.

69. Device according to one of claim 65, characterized in that the transmitted light measuring apparatus has more than one light source, wherein a main beam path of at least one of the light sources is arranged to run at a right angle to a detection sensor surface, and at least one further main beam path of at least one further light source is arranged to run at a different angle to a detection sensor surface.

70. Device according to one of claim 64, characterized in that a sensor unit, as seen looking in the axial retraction direction of the movable arm element, is arranged axially in front of a release unit for releasing a used sample carrier from the movable arm element.

71. Device according to one of claim 64, characterized by a sensor unit with a thermal sensor for detecting an excrement temperature, a body temperature, an excrement volume and/or an excrement volume flow.

72. Device for the on-site analysis of excrements, comprising a housing, a removal device, by means of which a sample of excrement can be removed, in which the removal device comprises an arm element for a sample carrier, which is axially movable relative to the housing and which is guided at least partially in a guide part in such a way that it can be retracted and extended, a feeding device, by means of which a new sample carrier can be provided for the removal device, and an analysis device, by means of which the removed sample can be at least partially analyzed, characterized in that the removal device comprises a release unit with stripping means for releasing a used sample carrier from the movable arm element.

73. Method for operating a device for the on-site analysis of excrements, in which a sample carrier is moved for sampling linearly relative to a sensor unit by means of an axially movable arm element, in which the sample carrier is held on a holder of the axially movable arm element, and in which a sample of excrement carried by the sample carrier is at least partially analyzed on the device, characterized in that a used sample carrier is released from the holder by means of the axial movement of the axially movable arm element in the axial movement direction.

74. Method for operating a device for the on-site analysis of excrements, in which a sample carrier is moved for sampling linearly relative to a sensor unit by means of an axially movable arm element, in which the sample carrier is held on a holder of the axially movable arm element, and in which a sample of excrement carried by the sample carrier is at least partially analyzed on the device, characterized in that a sample carrier provided with excrement is moved between an illumination device and a detection device and transilluminated for a transmitted light measurement by means of the illumination device.

75. Method according to claim 74, characterized in that the sample carrier provided with excrement is at rest or moved during the transmitted light measurement.

76. Method according to claim 74, characterized in that a moistening of the sample carrier with excrement, particularly with urine, is supported by means of additional movements, particularly in the axial extension and/or retraction direction, of the axially movable arm element.

77. Method according to claim 74, characterized in that excess excrement, particularly excess urine, is removed from the axially movable arm element, the sample carrier and/or the holder by a vibration of said axially movable arm element.

78. Arrangement consisting of a toilet, a urinal, or the like, and a device according to claim 64.

79. Sample carrier for the on-site analysis of excrements, comprising a main part, wherein the sample body has individual sample analysis fields with different geometrical shapes.

80. Sample carrier, particularly according to claim 79, for an on-site analysis of excrements, comprising a main part, wherein sample analysis fields are arranged both on the front side and on the rear side of the main part.

81. Sample carrier according to claim 79, characterized in that individual sample analysis fields have different indicators.

82. Sample carrier according to claim 79, characterized in that individual sample analysis fields are arranged on the main part at a distance from one another.

83. Sample carrier according to claim 79, characterized in that the main part and individual sample analysis fields are at least partially translucent.
